(19) United States
(12) Patent Application Publication
(10) Pub. No.: US 2016/0051150 A1
(43) Pub. Date: Feb. 25, 2016

(54) METHOD AND APPARATUS FOR MEASURING BLOOD PRESSURE USING AN ACOUSTIC SIGNAL

(71) Applicant: KONINKLIJKE PHILIPS N.V., Eindhoven (NL)

(72) Inventor: Ronaldus Maria AARTS, Geldrop (NL)

(21) Appl. No.: 14/825,387

(22) Filed: Aug. 13, 2015

(30) Foreign Application Priority Data
Aug. 22, 2014 (EP) 14182013.4

(51) Int. Cl. A61B 5/021; A61B 5/00; A61B 7/04

(52) U.S. Cl. CPC A61B 5/021; A61B 7/04; A61B 5/6817; A61B 5/7275; A61B 5/7278

(57) ABSTRACT

There is provided a method for measuring the blood pressure of a subject, comprising receiving an acoustic signal comprising an electronic representation of sounds generated by a hemodynamic effect of the subject's heart beats on one or more blood vessels in a body part of a subject; deriving one or more parameters from the received acoustic signal; and calculating a blood pressure value based on the one or more derived parameters.

### CROSS REFERENCE TO RELATED APPLICATIONS

This application claims the benefit of EP provisional application serial no. 14182013.4 filed Aug. 22, 2014, which is incorporated herein by reference.

### TECHNICAL FIELD OF THE INVENTION

The invention relates to a method and apparatus for measuring the blood pressure of a subject using an acoustic signal, and in particular relates to a method and apparatus for measuring the blood pressure of a subject using an acoustic signal acquired from within the subject's ear canal.

### BACKGROUND TO THE INVENTION

Blood pressure (BP), sometimes referred to as arterial blood pressure, is the pressure exerted by circulating blood upon the walls of blood vessels, and is one of the principal vital signs. When used without further specification, “blood pressure” usually refers to the arterial pressure of the systemic circulation. During each heart beat, blood pressure varies between a maximum (systolic) and a minimum (diastolic) pressure. The blood pressure in the circulation is principally due to the pumping action of the heart. Blood pressure control disorders include: high blood pressure, low blood pressure, and blood pressure that shows excessive or maladaptive fluctuation. Relationships between BP values and cardio vascular diseases and renal morbid- and fatal events have been shown by a large number of observational studies (see, e.g., G Mancia et. al.,“ESH/ESC Guidelines for the management of arterial hypertension”, 2013).

Arterial pressure is most commonly measured via a sphygmomanometer, which historically used the height of a column of mercury to reflect the circulating pressure. Consequently, blood pressure values are generally reported in millimeters of mercury (mmHg), even though modern aneroid and electronic devices do not contain mercury. In the clinic, non-invasive arterial blood pressure (NIBP) is measured by slowly varying the pressure in a cuff that is wrapped around the upper arm of a subject. The NIBP is determined either by measuring sound distal from the cuff (the auscultatory method, based on Korotkoff sounds) or by measuring pressure pulsations in the cuff caused by volume pulsations of the arm and brachial artery and extracting features from the envelope of these pressure pulses (the oscillometric method). The auscultatory method is the “gold standard” for cuff based NIBP measurements. The Korotkoff sounds, on which the auscultatory method is based, are generated by turbulence in the blood flow in the brachial artery, caused by the constriction of this artery by the inflated cuff. Normally (i.e. when no constriction is present) the blood flow in an artery is laminar. The Korotkoff sounds therefore only occur when the pressure in the cuff is lower than the systolic blood pressure (so that there is some blood flow in the artery) but higher than the diastolic blood pressure (so that the blood flow is turbulent rather than laminar). It will be appreciated that Korotkoff sounds are distinct from heart sounds (i.e. the noises generated by the closing of heart valves) and from the sounds generated by normal blood flow through unconstricted blood vessels. The generation of Korotkoff sounds requires the application of an external pressure to a blood vessel, and thus these sounds do not occur naturally and cannot be passively monitored.

It is known that there are problems with clinically acquired blood pressure measurements, such as the phenomenon of white coat hypertension (where subjects exhibit elevated blood pressure in a clinical setting but not in other settings). Ambulatory blood pressure devices (auscultatory or oscillometric), which are worn continuously by a subject and take readings at regular intervals (e.g. every half hour) throughout the day and night, exist and have been used for identifying and mitigating such measurement problems. However; ambulatory blood pressure monitoring using such devices requires the constant wearing of a cuff and an electronic pump. Furthermore, the device must be fitted by a healthcare professional and cannot be removed until the end of the monitoring period. Ambulatory blood pressure monitoring is therefore very intrusive into the subject's life.

Several non-cuff-based techniques for home BP measurement, which do not require professional fitting or continuous wearing of a blood pressure measurement device, have been developed. These techniques use the pulse wave velocity (PWV) principle and/or the related pulse transit times (PTT) principle, which relates the velocity at which an arterial pressure pulse travels along the arterial tree to the underlying blood pressure. Accordingly, after a calibration process (to account for the fact that blood pressure is also a function of arterial stiffness, which is unknown and highly nonlinear) these techniques can provide indirect estimates of blood pressure by translating PWV values into blood pressure values. The calibration process must be performed each time a measurement is acquired, which is inconvenient for the subject and can reduce compliance with a home monitoring program. Furthermore, all of these techniques suffer from at least one of the additional drawbacks of being obtrusive, expensive, and/or not accurate.

For example, WO 2007/023426 describes a PWV method where a transducer placed on the arm of a subject emits electromagnetic signals and the transducer and a further sensor placed at a different position on the arm detect reflections of these electromagnetic signals. The need for two transducers and for the active generation of a signal makes the required apparatus obtrusive and expensive.

As another example, US 2011/0196244 describes a blood pressure measurement technique which uses a single photoplethysmograph (PPG) sensor and derives the blood pressure using only the PPG waveform. However; optical methods like PPG can only measure the blood flow at a peripheral level (i.e. to the skin). Blood flow to the skin is a poor representation of arterial hemodynamics, and in some cases (e.g. in a cold environment) vasoconstriction occurs, which seriously hampers such optical methods. The accuracy of PPG-based blood pressure measurement techniques is therefore limited.

An accurate, inexpensive and unobtrusive means of measuring blood pressure, particularly a means which is suitable for home monitoring, would be a valuable tool to improve outcomes and quality of care for subjects with blood pressure disorders. There is therefore a need for an improved method and apparatus that can be used by a subject to quickly and easily obtain accurate measurements of their own blood pressure in a home environment.

### SUMMARY OF THE INVENTION

According to a first aspect of the invention, there is provided a method for measuring the blood pressure of a subject, comprising receiving an acoustic signal comprising an electronic representation of sounds generated by a hemodynamic effect of the subject's heart beats on one or more blood vessels in a body part of a subject; deriving one or more parameters from the received acoustic signal; and calculating a blood pressure value based on the one or more derived parameters.

Preferably the calculating uses one or more relationships between the derived parameters and a blood pressure value generated using a machine learning process.

In particular embodiments of the invention the method further comprises using a machine learning process to generate one or more relationships between the derived parameters and a blood pressure value, based on a historical data set of derived parameters and corresponding reference blood pressure values for a plurality of subjects.

In some embodiments of the invention the method further comprises receiving a reference blood pressure value for the subject; and updating the one or more relationships using the one or more derived parameters and the reference blood pressure value.

In some embodiments of the invention the received acoustic signal comprises a processed acoustic signal, and the method further comprises receiving a raw acoustic signal; and processing the raw acoustic signal to produce the processed acoustic signal. In some such embodiments the processing comprises one or more of amplifying, filtering and band-limiting the raw acoustic signal.

In preferred embodiments, the one or more parameters comprises at least one of:
the envelope of the acoustic signal waveform;
the ratio, p1/p2, of the height of a first peak, p1, in the acoustic signal to the height of a second peak, p2, in the acoustic signal;
the ratio, p1/pn, of p1 to the height of a further peak, pn, in the acoustic signal;
the area under the curve, AUC, of the envelope of the acoustic signal waveform;
the slew rate of the peaks in the acoustic signal;
the period, T, of the heart beat signal;
p1/(AUC/T);
p2/(AUC/T).

In some such embodiments the one or more parameters comprises the area under the curve (AUC) of the envelope of the acoustic signal waveform; and the period (T) of the heart beat signal.

In preferred embodiments of the invention the calculated blood pressure value comprises one or more of the diastolic blood pressure, the systolic blood pressure, and the mean blood pressure.

Preferably, the received acoustic signal was measured by an in-ear microphone and the body part comprises the ear canal.

There is also provided, according to a second aspect of the invention, a computer program product, comprising computer readable code embodied therein, the computer readable code being configured such that, on execution by a suitable computer or processor, the computer or processor performs the method of the first aspect.

There is also provided, according to a third aspect of the invention, an apparatus for use in measuring the blood pressure of a subject, comprising a processing unit configured to perform the method of the first aspect.

In preferred embodiments of the invention the processing unit comprises a machine learning module for generating one or more relationships between the derived parameters and a blood pressure value, based on a historical data set of derived parameters and corresponding reference blood pressure values for a plurality of subjects.

There is also provided, according to a fourth aspect of the invention, a system for use in measuring the blood pressure of a subject, comprising an acoustic sensor configured to measure an acoustic signal containing sounds generated by a hemodynamic effect of the subject's heart beats on one or more blood vessels in a body part of a subject; and an apparatus according to the third aspect.

In particular embodiments of the invention the acoustic sensor comprises an in-ear microphone.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is an illustration of an apparatus for measuring the blood pressure of a subject according to an embodiment;

FIG. 2 is a flow chart illustrating a method of measuring the blood pressure of a subject according to a general embodiment of the invention; and

FIG. 3 is a graph of amplitude against time for an example received acoustic signal.

### DETAILED DESCRIPTION OF THE PREFERRED EMBODIMENTS

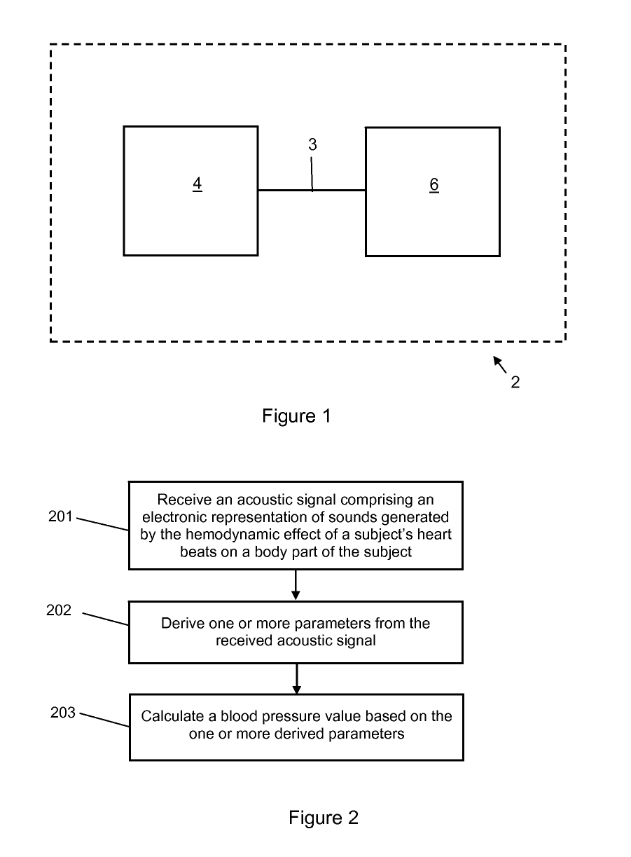

FIG. 1 shows an apparatus for measuring the blood pressure of a subject (patient) that can implement the method according to the invention. The apparatus 2 comprises a microphone 4, and a processing unit 6 that is in communication with the microphone 4 via a communications link 3, such that it can receive an acoustic signal from the microphone. The processing unit 6 is configured to calculate a blood pressure value using the received acoustic signal.

The microphone 4 is configured to acquire an acoustic signal which contains sounds generated by the hemodynamic effect of the subject's heart beats on one or more blood vessels in a body part of the subject. Normal blood flow through a blood vessel generates sound, e.g. through the interaction of the blood with the vessel walls. The amplitude of the generated sound increases with flow rate, and is therefore modulated by the subject's heart beat. A condition related to hemodynamic generation of sound is pulsatile tinnitus, in which a subject can hear a rhythmical noise having the same rate as their heart. Pulsatile tinnitus is caused by increased or changed blood flow in the vessels near the ear. Hemodynamically generated sounds are distinct, in both cause and nature, from heart sounds, which are generated by the closing of heart valves.

In preferred embodiments the microphone 4 is configured to be placed at least partly in one of the subject's ear canals. In such embodiments the acoustic signal acquired by the microphone contains sounds generated by the hemodynamic effect of the subject's heart beats on one or more blood vessels in or near the ear in which the microphone 4 is placed. The blood vessels from which sounds are detectable by an in-ear microphone may include large arteries and veins in the neck and/or base of the skull (such as the carotid artery), and/or and smaller blood vessels in the ear itself. In some such embodiments the microphone is configured to be placed in the entrance to the ear canal, whilst a small flexible tube attached to the microphone extends fully into the ear canal. In some embodiments the microphone 4 is integrated into a headphone/earphone. In some embodiments two in-ear microphones are provided to simultaneously measure an acoustic signal in each ear of the subject, which can advantageously improve the signal to noise ratio (SNR). Alternatively or additionally, active noise cancelation techniques can be used to decrease the influence of external acoustical noises and hence improve the SNR.

It will be appreciated that, although preferred embodiments use an in-ear microphone, any microphone suitable for acquiring an acoustic signal which contains sounds generated by the hemodynamic effect of the subject's heart beats on one or more blood vessels in a body part of the subject could be used. For example, a wrist-mounted microphone could be used to detect sounds from the radial artery. Because hemodynamically generated sounds occur naturally, during normal operation of the circulatory system, this advantageously means that no additional equipment or techniques are required in order to measure the sounds (unlike, e.g., Korotkoff sounds, which require the application of an external pressure). Since only a microphone and a processing unit are required, apparatus suitable for implementing the invention can be very small and/or inexpensive.

In some embodiments the microphone is configured to begin measuring in response to receiving a start signal, e.g. from the processing unit 6 or from a manually operated switch comprised in the microphone 4. In some embodiments the microphone is configured to measure an acoustic signal for a predefined time period, which is preferably long enough to include at least three heart beats. In preferred embodiments the duration of the predefined time period is between 3 and 10 seconds. In some embodiments the microphone 4 is configured to measure an acoustic signal until it receives a stop signal, e.g. from the processing unit 6 or from a manually operated switch comprised in the microphone 4. In some embodiments the microphone 4 includes an integrated amplifier. It will be appreciated that the invention can be implemented using any small microphone having a good signal to noise ratio.

In preferred embodiments the processing unit 6 includes a signal processing module comprising an amplifier and a filter. The signal processing module is configured to generate a processed acoustic signal by amplifying, filtering and band-limiting a raw acoustic signal received from the microphone 4.

In some embodiments the processing unit 6 includes a machine learning module, configured to use standard machine learning techniques to identify or generate rules, relationships, etc. relating blood pressure to acoustic signal parameters.

In some embodiments the microphone 4 and the processing unit 6 are provided in a single device. In other embodiments the processing unit 6 is separate from the microphone 4. In such embodiments the microphone 4 and the processing unit 6 each include a communications interface to enable a communications link (which may be wired or wireless) to be established between the microphone 4 and the processing unit 6. In such embodiments the microphone 4 is configured to transmit data, e.g. a measured acoustic signal, to the processing unit 6. In some embodiments the processing unit 6 can also send control signals (e.g. an instruction to begin measuring) to the microphone 4.

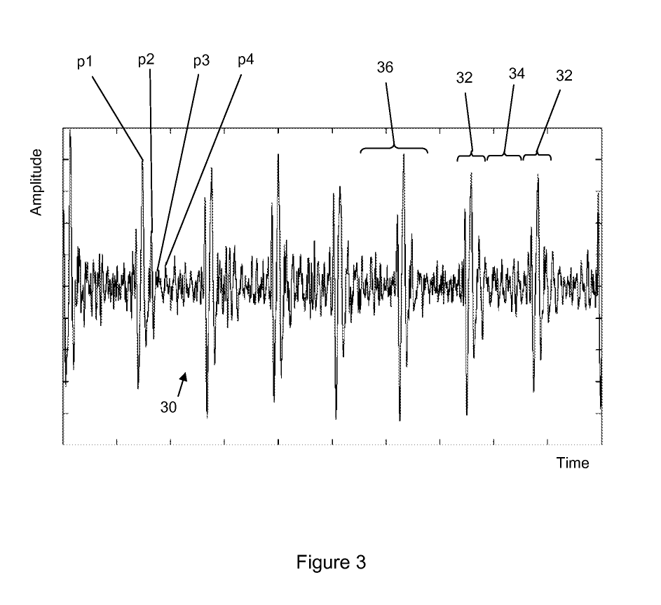

In preferred embodiments the processing unit 6 is configured to calculate a blood pressure value by performing the method shown in FIG. 2, which will now be explained.

FIG. 2 shows a method for measuring the blood pressure of a subject. In step 201 an acoustic signal (e.g. generated by the microphone 4) comprising an electronic representation of sounds generated by the hemodynamic effect of the subject's heart beat on one or more blood vessels in a body part of the subject is received (e.g. by the processing unit 6). In preferred embodiments the acoustic signal is generated by an in-ear microphone and the received acoustic signal comprises an electronic representation of sounds generated by the hemodynamic effect of the subject's heart beats on the carotid artery and/or adjacent blood vessels. In some embodiments the method includes the optional additional step (not illustrated) of triggering the microphone to begin measuring an acoustic signal (e.g. by the processing unit 6 sending a start signal to the microphone 4, or the activation of a start switch comprised in the microphone 4). In preferred embodiments the received acoustic signal includes at least three heart beats, and therefore covers a time period of at least 3 seconds.

In preferred embodiments the processing unit receives a raw (i.e. unprocessed) acoustic signal from the microphone, and the method includes the optional additional step of processing (e.g. with a signal processing module of the processing unit) the raw acoustic signal by amplifying, filtering, and band-limiting it to produce a processed acoustic signal. In some such embodiments the resulting processed acoustic signal is band-limited to, e.g., 35 Hz (although it will be appreciated that other frequencies could be used). As a result of the processing, any motion artefacts present in the raw signal (e.g. resulting from the subject moving whilst the microphone was measuring the acoustic signal) are removed. In embodiments where a raw acoustic signal is processed to generate a processed acoustic signal, the received acoustic signal of FIG. 2 comprises the processed acoustic signal. FIG. 3 shows a graph of signal amplitude against time for an exemplary received acoustic signal 30 generated by an in-ear microphone. The signal shown in FIG. 3 includes seven complete heart beats, during a period of just over eight seconds. Each heart beat is characterised by a set of relatively high peaks 32. Meanwhile, the periods between heart beats are characterized by relatively low peaks 34. A complete cardiac cycle 36 comprises a heart beat 32 and an intervening period 34.

In step 202 one or more parameters are derived (e.g. by the processing unit) from the received acoustic signal 30. Parameters which can be derived include:
The envelope (not shown) of the acoustic signal waveform;
the ratio (p1/p2) of the first peak (p1) to the second peak (p2) (i.e. the next adjacent peak);
the ratio (p1/pn) of the first peak to at least one further (i.e. third (p3), fourth (p4), nth (pn), etc.) peak;
the area under the curve (AUC) of the envelope;
the slew rate (i.e. slope divided by time progress, a measure of how quickly the peak height changes for a given set of peaks) of the peaks;
the period (T) of the heart signal (i.e. the number of beats in a given time frame);
p1/(AUC/T);
p2/(AUC/T).

Various techniques suitable for extracting each of the above parameters from an acoustic signal are known in the art. In preferred embodiments, all of the above parameters are derived. However, embodiments are possible in which only a subset of the above parameters are derived. For example, in some embodiments only AUC and T are derived. In some embodiments the ratio p1/p2 is calculated for each pair of adjacent peaks in the signal 30. In other embodiments the ration p1/p2 is only calculated for some pairs of adjacent peaks (e.g. the pairs comprising the highest peak for each heart beat and the next peak). In some embodiments each parameter is derived for a single cardiac cycle 36. In some such embodiments, a value of each parameter is derived for each complete cardiac cycle represented in the acoustic signal 30. In some such embodiments an overall average value for each parameter is calculated, using the values for each individual cardiac cycle.

In step 203 a blood pressure value is calculated based on the one or more derived parameters. In preferred embodiments the calculation involves inputting the derived parameters to a machine learning module (e.g. of the processing unit 6) which has been trained using acoustic signals and reference blood pressure data (e.g. acquired by the gold standard auscultatory method) for a large number of subjects. The machine learning module then calculates a blood pressure value using the derived parameters, based on, e.g. rules, relationships etc. identified or generated by the machine learning module during the training phase.

During the training phase the machine learning module uses standard machine learning techniques to identify or generate rules, relationships, etc. relating blood pressure to parameters of the acoustic signal. For example, in some embodiments a linear discriminant (LD) classifier, with low, normal and high classes (as described in R. O. Duda et. al.; Pattern Classification, 2nd ed.; Reading, Mass., USA; Wiley; 2001) is used.

In some embodiments the training phase is completed before the machine learning module is used in the measurement of a subject's blood pressure. For example, the training phase may be completed at a manufacturer's site, during manufacture and set-up of the processing unit. Alternatively, the training of the machine learning module may be updated whilst the processing unit is in use, when reference BP data for a subject becomes available, e.g. because the subject's blood pressure has been measured clinically.

It will be appreciated that the calculating step could be performed using other suitable techniques known in the art, for example techniques based on neural nets.

The calculated blood pressure value may comprise any or all of a systolic blood pressure, a mean blood pressure and/or a diastolic blood pressure. In preferred embodiments, all of a systolic blood pressure, a mean blood pressure and a diastolic blood pressure are calculated based on the one or more derived parameters.

There is therefore provided a method and apparatus that enable the accurate home-based measurement of blood pressure in a convenient and unobtrusive way, using simple and inexpensive equipment. Embodiments of the invention enable hemodynamic information—including blood pressure—to be derived from an acoustic pulse measured by a single in-ear microphone, without requiring an external pressure to be applied to a blood vessel, without requiring any other sensors, and without using PTT or PWV principles. Furthermore, because the acoustic pulse in the ear is generated, at least partly, by blood flowing through large, central blood vessels such embodiments can be more accurate than prior art techniques which measure only peripheral hemodynamics.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method for measuring the blood pressure of a subject, comprising:
receiving an acoustic signal comprising an electronic representation of sounds generated by a hemodynamic effect of the subject's heart beats on one or more blood vessels in a body part of a subject;
deriving one or more parameters from the received acoustic signal; and
calculating a blood pressure value based on the one or more derived parameters.

2. The method according to claim 1, wherein the calculating uses one or more relationships between the derived parameters and a blood pressure value generated using a machine learning process.

3. The method according to claim 2, further comprising using a machine learning process to generate one or more relationships between the derived parameters and a blood pressure value, based on a historical data set of derived parameters and corresponding reference blood pressure values for a plurality of subjects.

4. The method according to claim 2, further comprising:
receiving a reference blood pressure value for the subject; and
updating the one or more relationships using the one or more derived parameters and the reference blood pressure value.

5. The method according to claim 1, wherein the received acoustic signal comprises a processed acoustic signal, the method further comprising:
receiving a raw acoustic signal; and
processing the raw acoustic signal to produce the processed acoustic signal.

6. The method according to claim 5, wherein the processing comprises one or more of amplifying, filtering and band-limiting the raw acoustic signal.

7. The method according to claim 1, wherein the one or more parameters comprises at least one of:
the envelope of the acoustic signal waveform;
the ratio, p1/p2, of the height of a first peak, p1, in the acoustic signal to the height of a second peak, p2, in the acoustic signal, wherein p2 is the next adjacent peak to p1;
the ratio, p1/pn, of p1 to the height of a further peak, pn, in the acoustic signal;
the area under the curve, AUC, of the envelope of the acoustic signal waveform;
the slew rate of the peaks in the acoustic signal;
the period, T, of the heart beat signal;
p1/(AUC/T);
p2/(AUC/T).

8. The method according to claim 7, wherein the one or more parameters is a plurality of parameters comprising:
the area under the curve, AUC, of the envelope of the acoustic signal waveform; and
the period, T, of the heart beat signal.

9. The method according to claim 1, wherein the calculated blood pressure value comprises one or more of the diastolic blood pressure, the systolic blood pressure, and the mean blood pressure.

10. The method according to claim 1, wherein the received acoustic signal was measured by an in-ear microphone and the body part comprises the ear canal.

11. A computer program product, comprising computer readable code embodied therein, the computer readable code being configured such that, on execution by a suitable computer or processor, the computer or processor performs the method described in claim 1.

12. An apparatus for use in measuring the blood pressure of a subject, comprising a processing unit configured to perform the method of claim 1.

13. The apparatus of claim 12, wherein the processing unit comprises a machine learning module for generating one or more relationships between the derived parameters and a blood pressure value, based on a historical data set of derived parameters and corresponding reference blood pressure values for a plurality of subjects.

14. A system for use in measuring the blood pressure of a subject, comprising:
an acoustic sensor configured to measure an acoustic signal containing sounds generated by a hemodynamic effect of the subject's heart beats on one or more blood vessels in a body part of a subject; and
an apparatus according to claim 12.

15. A system according to claim 14, wherein the acoustic sensor comprises an in-ear microphone.
